(19) United States
(12) Patent Application Publication
(10) Pub. No.: US 2013/0178808 A1
(43) Pub. Date: Jul. 11, 2013

(54) Body Fluid Sucking Device

(71) Applicant: Chih Ming Chen, New Taipei City (TW)

(72) Inventor: Chih Ming Chen, New Taipei City (TW)

(73) Assignee: AVITA CORPORATION, New Taipei City (TW)

(21) Appl. No.: 13/539,485

(22) Filed: Jul. 1, 2012

(30) Foreign Application Priority Data
Jul. 1, 2011 (TW) 100212104

(51) Int. Cl. A61M 1/00

(52) U.S. Cl. CPC A61M 1/0023
USPC 604/319

(57) ABSTRACT

A body fluid sucking device includes a body fluid collecting container, a sucking unit, an air extracting unit, and a filtration member. The sucking unit is coupled with the body fluid collecting container and has a first inner channel interlinked with an inner space of the body fluid collecting container. The air extracting unit is coupled with the body fluid collecting container and includes a second inner channel having a first opening end interlinked with the inner space of the body fluid collecting container, and a second opening end. The filtration member, which covers the first opening end or the second opening end, has a multi-aperture structure with waterproof and air-permeable effects. When air in the body fluid collecting container is extracted out through the air extracting unit, the air and the body fluid are both filtrated by the filtration member which blocks the body fluid (such as water, snot, snivel, spittle, and etc.) but allows the air to pass through, so as to prevent the body fluid from getting into the air extracting unit and resulting in a cross-infection, as well as to prevent the body fluid from getting into an air extracting apparatus (such as an air pump) through the air extracting unit and causing damages to the body fluid sucking device.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

1. Field of Invention

The present invention relates to a body fluid sucking device, and more particularly to a body fluid sucking device comprising a sucking unit adapted for having contact with a body of a subject at an end thereof, and an air extracting unit operated by a guardian of the subject for extracting air therethrough, so that body fluid of the subject is sucked out and collected.

2. Description of Related Arts

A child who cannot take care of himself/herself or a patient in need of nursing care may not be able to blow his/her nose, so that his/ her nasal cavity may be blocked with snivel that causes a difficulty in breathing. A traditional nose suction device on sale in the market generally has a snivel collecting container, a cover body screwed with the snivel collecting container, a sucking tube and an air extracting tube respectively provided on the cover body. When in use, an end of the sucking tube is inserted into a nostril and the treatment personnel extracts air through the air extracting tube, so as to suck out the snivel. However, in the conventional nose suction device, the sucking tube, the snivel collecting container, and the air extracting tube has a connecting channel, so that when the nose suction device is in operation to suck snivel for a patient, a child or a senior, the snivel is easy to be sucked into the air pump through the air extracting tube and may cause a damage of the air pump, or the treatment personnel may have contact with the snivel and result in a risk of cross-infection.

In view of the above, the inventor herein who has many years of experience in manufacturing, developing and designing the related products has developed a practical body fluid sucking device of the present invention after an advance design and an attentive evaluation.

### SUMMARY OF THE PRESENT INVENTION

A main object of the present invention is to provide a body fluid sucking device for solving the problem that when the nose suction device being in operation to suck snivel for a patient, a child or a senior, the snivel is easy to be sucked into the air pump through the air extracting tube that causes damage to the air pump, or that the treatment personnel may have contact with the snivel and result in a risk of cross-infection.

Additional advantages and features of the invention will become apparent from the description which follows, and may be realized by means of the instrumentalities and combinations particular point out in the appended claims.

According to the present invention, the foregoing and other objects and advantages are attained by a body fluid sucking device comprising a body fluid collecting container, a sucking unit, an air extracting unit, and a filtration member. The sucking unit is coupled with the body fluid collecting container and has a first inner channel interlinked with an inner space of the body fluid collecting container. The air extracting unit is coupled with the body fluid collecting container and includes a second inner channel having a first opening end, which is interlinked with the inner space of the body fluid collecting container, and a second opening end. The filtration member can be embodied as a filtering membrane covered at the first opening end or the second opening end and has a multi-aperture structure with waterproof and air-permeable effects.

The present invention is advantageous in that it provides the filtration member covered at the opening end of the inner channel of the air extracting unit, so that when air in the body fluid collecting container is extracted out through the air extracting unit, the air and the body fluid are both filtrated by the filtration member which blocks the body fluid (such as water, snot, snivel, spittle, and etc.) but allows the air to pass through, so as to prevent the body fluid from getting into the air extracting unit and resulting in a cross-infection, as well as to prevent the body fluid from getting into an air extracting apparatus (such as an air pump) through the air extracting unit and causing damages to the body fluid sucking device.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a partial schematic view of a body fluid sucking device according to a preferred embodiment of the present invention.

FIG. 2 is a partial schematic view of a body fluid sucking device according to a second preferred embodiment of the present invention.

FIG. 3 is a partial schematic view of a body fluid sucking device according to a third preferred embodiment of the present invention.

FIG. 4 is a sectional view of a body fluid sucking device according to a fourth preferred embodiment of the present invention.

FIG. 5 is a sectional view of a body fluid sucking device according to a fifth preferred embodiment of the present invention.

FIG. 6 is a partial exploded perspective view illustrating a connection between an air extracting unit and a movable retaining sleeve according to the above fifth preferred embodiment of the present invention.

FIG. 7 is an elevation view illustrating a configuration of the body fluid sucking device according to the above fifth preferred embodiment of the present invention.

### DETAILED DESCRIPTION OF THE PREFERRED EMBODIMENT

The following description is disclosed to enable any person skilled in the art to make and use the present invention. Preferable embodiments are provided in the following description only as examples and modifications will be apparent to those skilled in the art. The general principles defined in the following description would be applied to other embodiments, alternatives, modifications, equivalents, and applications without departing from the spirit and scope of the present invention.

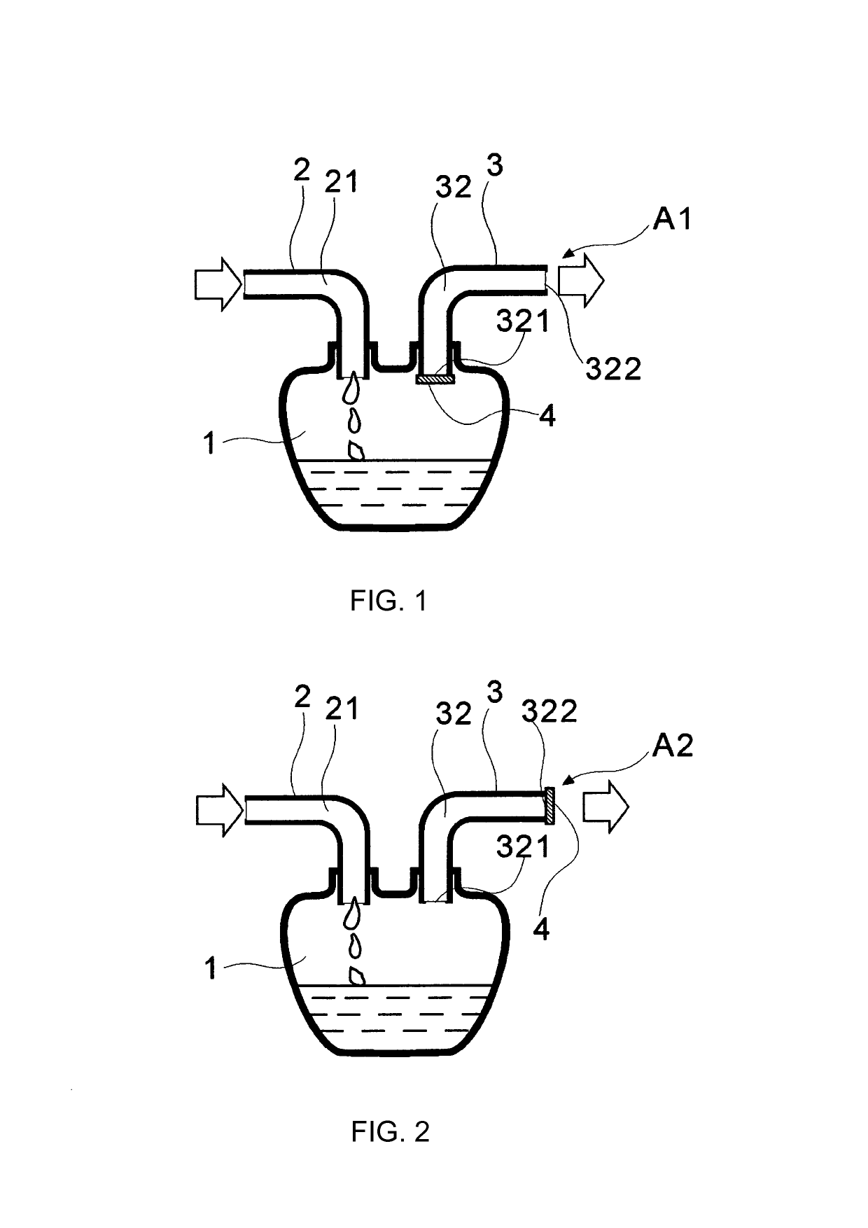

Referring to FIG. 1 of the drawings, a body fluid sucking device Al according to a preferred embodiment of the present invention comprises a body fluid collecting container 1, a sucking unit 2, an air extracting unit 3, and a filtration member 4. The sucking unit 2, which is coupled with the body fluid collecting container 1, has a first inner channel 21 interlinked with an inner space of the body fluid collecting container 1. The air extracting unit 3, which is coupled with the body fluid collecting container 1, has a second inner channel 32 having a first opening end 321 and a second opening end 322. The first opening end 321 is interlinked with the inner space of the body fluid collecting container 1. The filtration member can be embodied as a filtering membrane, having a multi-aperture structure with waterproof and air-permeable effects, covered at the first opening end 321. When inner air in the body fluid collecting container 1 is extracted out through the air extracting unit 3, the air and the body fluid are both filtrated by the filtration member 4 which blocks the body fluid (such as water, snot, snivel, spittle, and etc.) but allows the air to pass therethough.

Referring to FIG. 2 of the drawings, a body fluid sucking device A2 according to a second preferred embodiment of the present invention has a similar structure with the body fluid sucking device Al according to the above first preferred embodiment of the present invention except that the filtration member 4 is provided to cover the second opening end 322 for providing the same function of blocking the body fluid (such as water, snot, snivel, spittle, and etc.) but allowing the air to pass therethough.

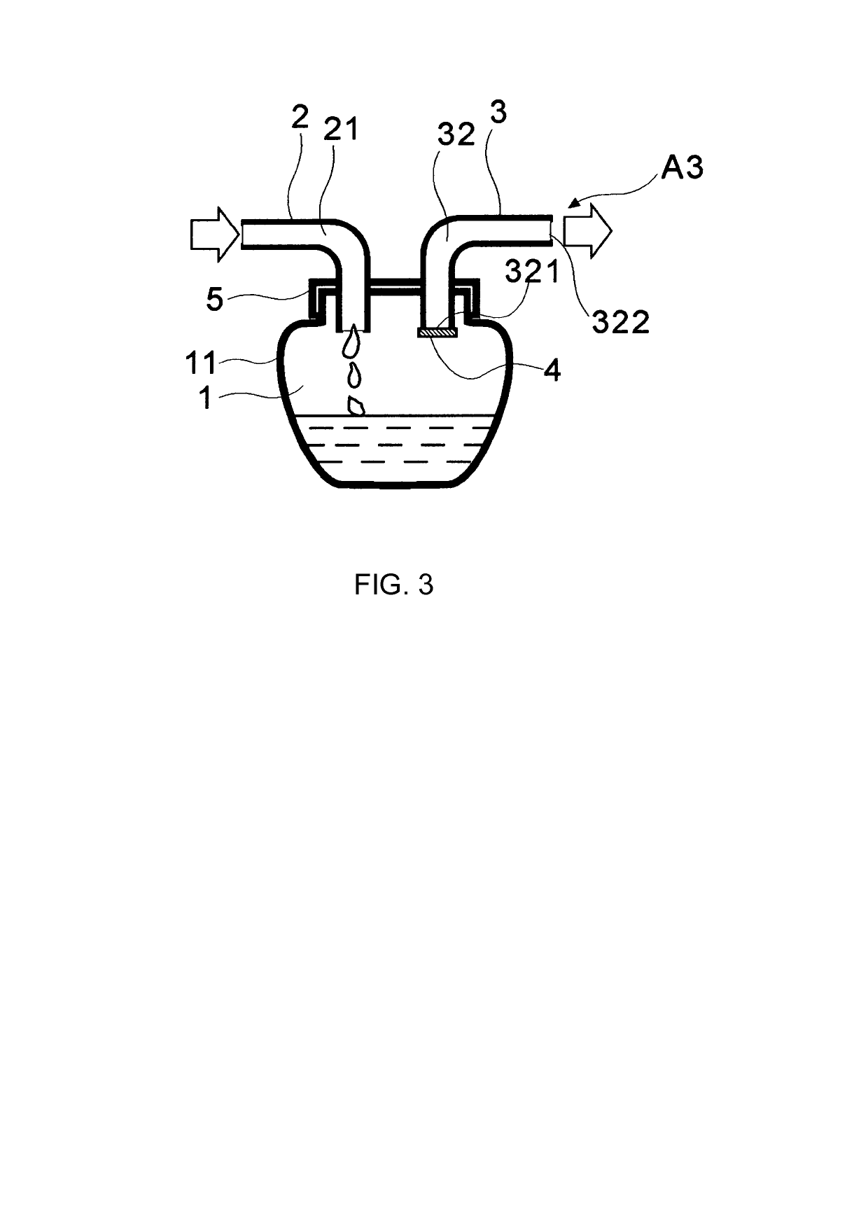

Referring to FIG. 3 of the drawings, a body fluid sucking device A3 according to a third preferred embodiment of the present invention has a similar structure with the body fluid sucking device Al according to the above first preferred embodiment of the present invention except that the body fluid collecting container 1 of this third preferred embodiment further comprises a cap 5 and a body fluid collecting cup 11 which are connected or detachably coupled with each other. When the cap 5 and the body fluid collecting cup 11 are detached from the body fluid collecting container 1, the body fluid collected therein can be cleaned up, and the filtration member 4 also can be cleaned or replaced.

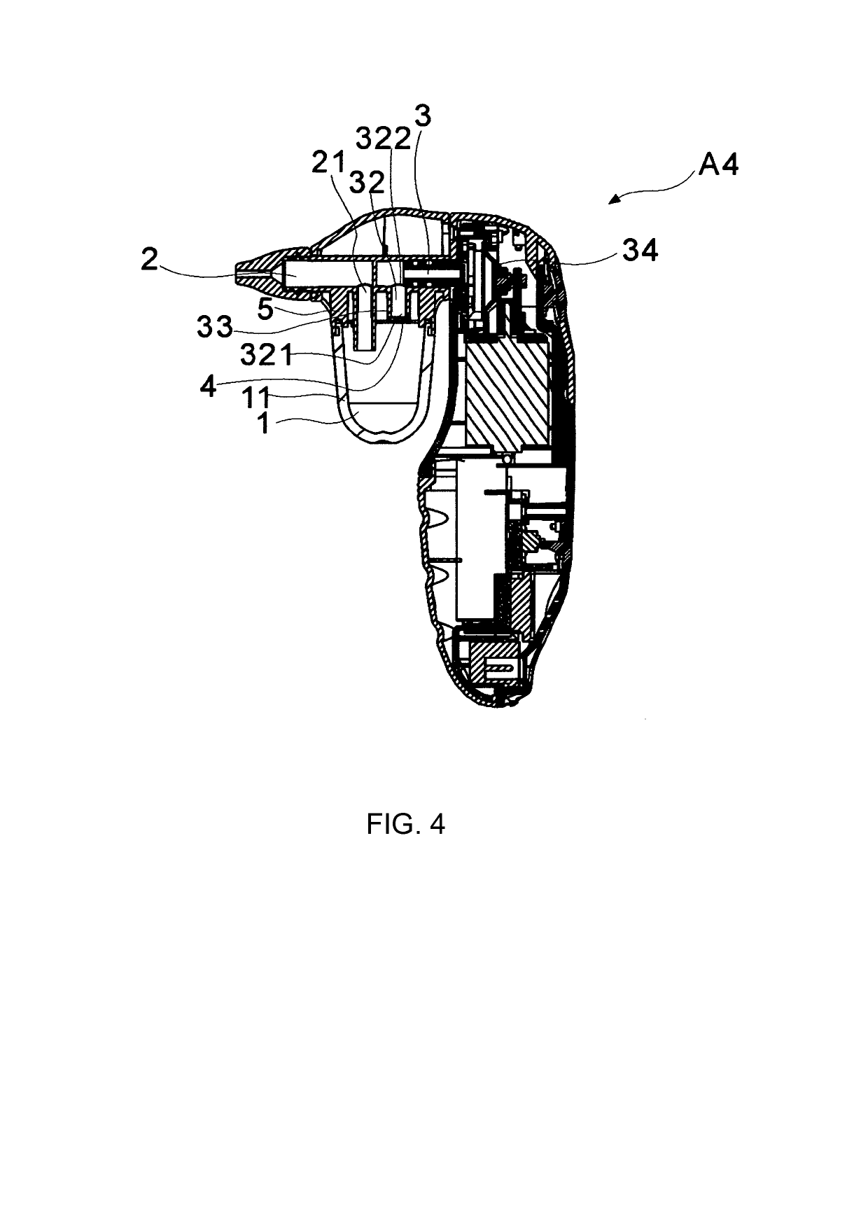

Referring to FIG. 4 of the drawings, a body fluid sucking device A4 according to a fourth preferred embodiment of the present invention has a similar structure with the body fluid sucking device A3 according to the above third preferred embodiment of the present invention except that the cap 5 can be integrally connected with the sucking unit 2 and the air extracting unit 3. According to this fourth preferred embodiment, the air extracting unit 3 further comprises an air extracting apparatus 34 interlinked with the second opening end 322. The air extracting apparatus 34 can be but not limited to an electrical pump.

According to this fourth preferred embodiment, the air extracting unit 3 comprises a tube structure 33 protruded into the inner space of the body fluid collecting container 1. The filtration member 4 covers a first opening end 321 of the tube structure 33 facing towards the inner space of the body fluid collecting container 1.

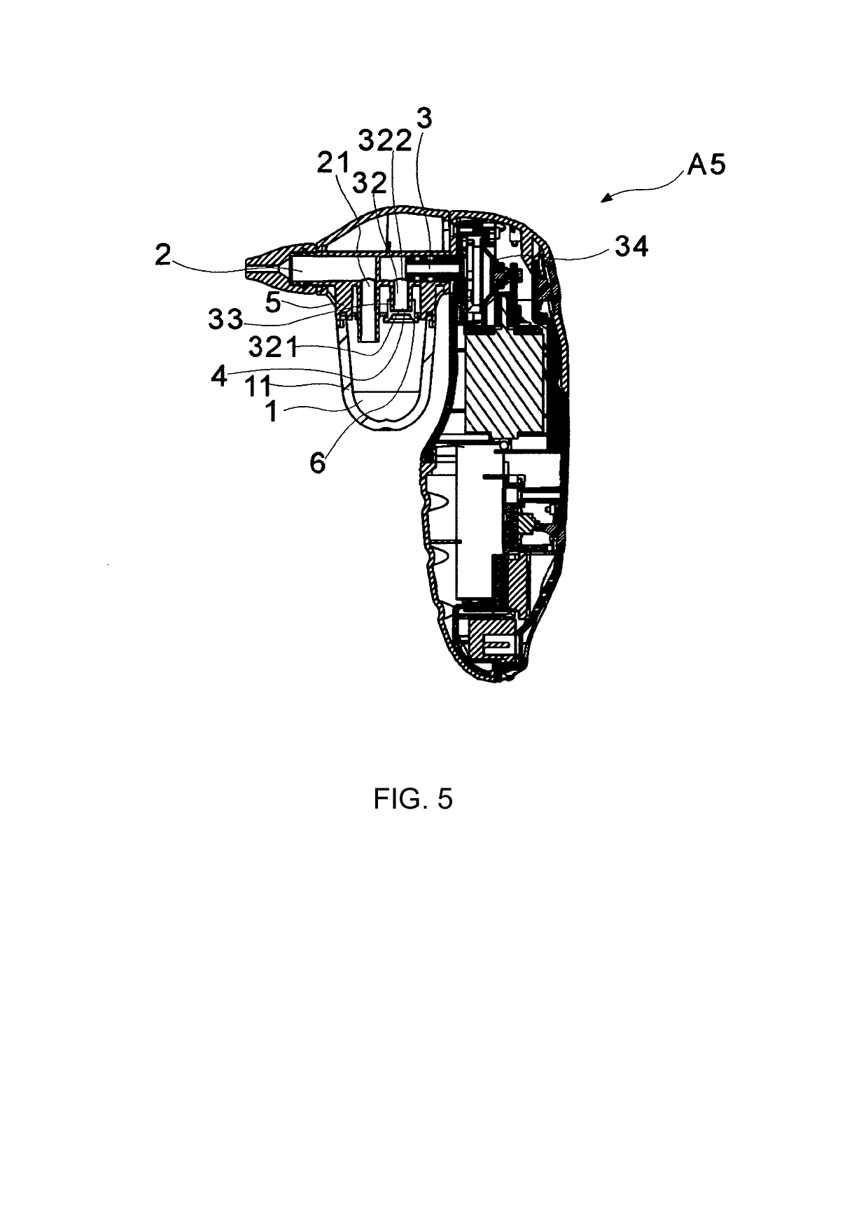

Referring to FIG. 5 of the drawings, a body fluid sucking device AS according to a fifth preferred embodiment of the present invention has a similar structure with the body fluid sucking device A4 according to the above fourth preferred embodiment of the present invention except that the body fluid collecting unit 1 is further provided with a retaining sleeve 6 through which the filtration member 4 covers the first opening end 321 of the tube structure 33 facing towards the inner space of the body fluid collecting container 1. When the filtration member 4 is used for a relatively long period of time or clogged, the retaining sleeve 6 can be detached so that the filtration member 4 can be cleaned or replaced.

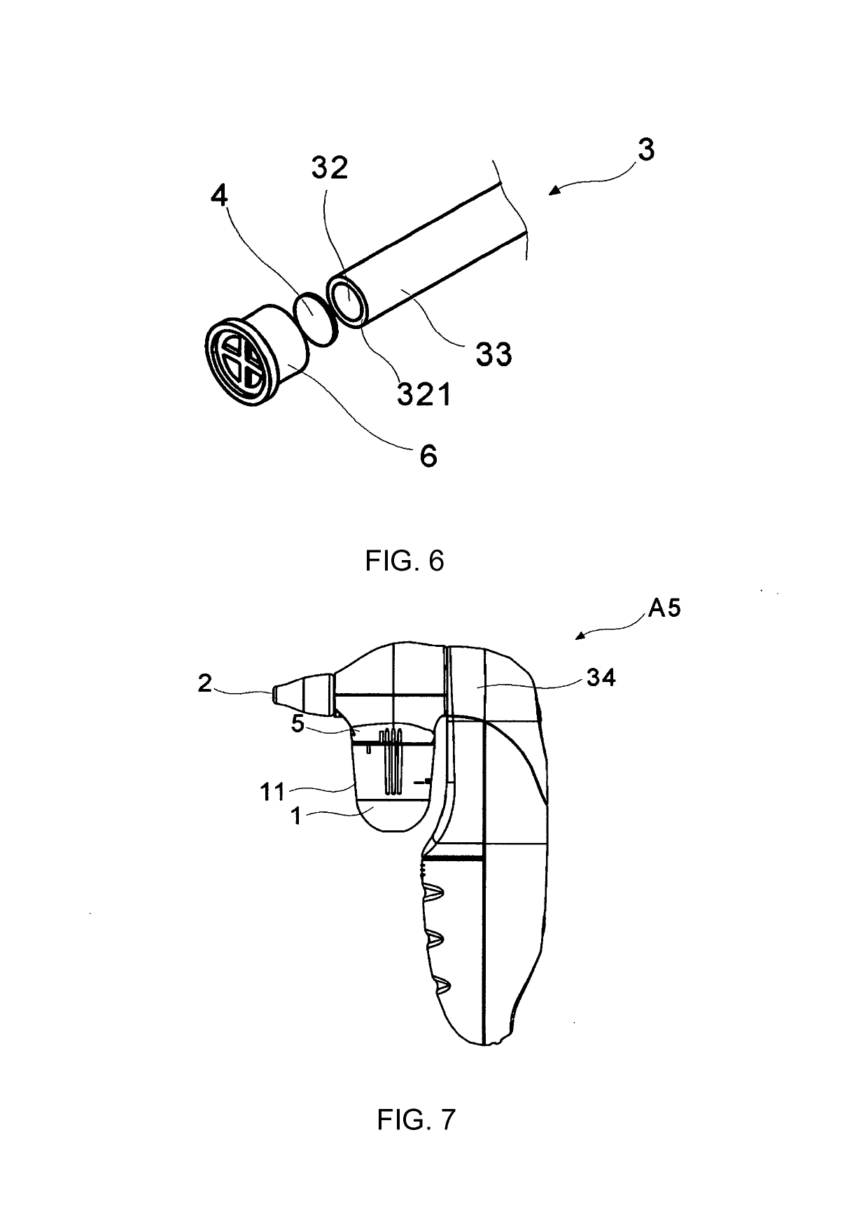

Referring to FIG. 6 of the drawings, the filtration member 4 can be provided in the retaining sleeve 6 so that the filtration member 4 is secured at the first opening end 321 of the tube structure 33 facing towards the inner space of the body fluid collecting container 1. When the filtration member 4 has been used for a relatively long period of time or clogged, the retaining sleeve 6 can be detached so that the filtration member 4 in the retaining sleeve can be cleaned or replaced with a new retaining sleeve 6 installed with a new filtration member 4.

An integral configuration of the body fluid sucking device AS according to the fifth preferred embodiment of the present invention is shown in FIG. 7 of the drawings. The body fluid sucking device, which is electrically driven, comprises an air extracting apparatus 34 (electrical pump) connected with the air extracting unit 3. When an end of the air sucking unit 2 has contacted with a human body and a treatment personnel actuates the air extracting apparatus 34 (electrical pump), the body fluid is sucked out and collected.

The filtration member 4 in the fifth preferred embodiments above has a multi-aperture structure with an aperture diameter smaller than 100 μm (a multi-aperture structure with a preferred diameter of the aperture ranged from 0.01 μm to 10 μm). The filtration member 4 is made of material selected from the group consisting of poly tetra fluoro ethylene (PTFE), expended poly tetra fluoro ethylene (ePTFE), thermoplastic polyurethane (TPU), and polyethylene (PE). The aperture diameter of the filtration member 4 is selected in consideration of preventing water drop (such as rain, fog, etc.) with a diameter larger than about 100 μm from passing therethrough whilst allowing a molecule of water vapor with a diameter ranged from about 0.01 μm to 10 μm to pass therethrough, so that its waterproof and air-permeable effects can thus be achieved. In other words, the body fluid (such as water, snot, snivel, spittle, and etc.) is blocked whilst air is allowed to pass therethrough.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

1. A body fluid sucking device, comprising:
a body fluid collecting container;
a sucking unit coupled with said body fluid collecting container, wherein said sucking unit includes a first inner channel interlinked with an inner space of said body fluid collecting container;
an air extracting unit coupled with said body fluid collecting container, wherein said air extracting unit includes a second inner channel, which includes a first opening end interlinked with said inner space of said body fluid collecting container, and a second opening end; and
a filtration member covered at one of said first opening end and said second opening end, wherein said filtration member has a multi-aperture structure with waterproof and air-permeable effects.

2. The body fluid sucking device, as recited in claim 1, wherein said body fluid collecting container comprises a cap and a body fluid collecting cup which are connected or detachably coupled with each other.

3. The body fluid sucking device, as recited in claim 2, wherein said cap is integrated with said sucking unit and said air extracting unit to form a one piece structure.

4. The body fluid sucking device, as recited in claim 1, wherein said air extracting unit further comprises an air extracting apparatus interlinked with said second opening end.

5. The body fluid sucking device, as recited in claim 1, wherein said air extracting unit comprises a tube structure protruded into said inner space of said body fluid collecting container , wherein said filtration member covers said first opening end of said tube structure facing towards said inner space of said body fluid collecting container.

6. The body fluid sucking device, as recited in claim 5, further comprising a retaining sleeve through which said filtration member covers said first opening end of said tube structure facing towards said inner space of said body fluid collecting container.

7. The body fluid sucking device, as recited in claim 5, further comprising a retaining sleeve combined with said filtration member, so that said filtration member is secured at said first opening end of said tube structure facing towards said inner space of said body fluid collecting container.

8. The body fluid sucking device, as recited in claim 1, wherein said filtration member has a multi-aperture structure with an aperture diameter smaller than 100 μm.

9. The body fluid sucking device, as recited in claim 1, wherein said filtration member has a multi-aperture structure with an aperture diameter ranged from 0.01 μm to 10 μm.

10. The body fluid sucking device, as recited in claim 1, wherein said filtration member is made of material selected from a group consisting of poly tetra fluoro ethylene (PTFE), expended poly tetra fluoro ethylene (ePTFE), thermoplastic polyurethane (TPU), and polyethylene.

11. The body fluid sucking device, as recited in claims 2, wherein said filtration member has a multi-aperture structure with an aperture diameter smaller than 100 μm.

12. The body fluid sucking device, as recited in claims 3, wherein said filtration member has a multi-aperture structure with an aperture diameter smaller than 100 μm.

13. The body fluid sucking device, as recited in claims 4, wherein said filtration member has a multi-aperture structure with an aperture diameter smaller than 100 μm.

14. The body fluid sucking device, as recited in claims 5, wherein said filtration member has a multi-aperture structure with an aperture diameter smaller than 100 μm.

15. The body fluid sucking device, as recited in claims 6, wherein said filtration member has a multi-aperture structure with an aperture diameter smaller than 100 μm.

16. The body fluid sucking device, as recited in claims 7, wherein said filtration member has a multi-aperture structure with an aperture diameter smaller than 100 μm.

17. The body fluid sucking device, as recited in any one of claims 2, wherein said filtration member has a multi-aperture structure with an aperture diameter ranged from 0.01 μm to 10 μm.

18. The body fluid sucking device, as recited in any one of claims 3, wherein said filtration member has a multi-aperture structure with an aperture diameter ranged from 0.01 μm to 10 μm.

19. The body fluid sucking device, as recited in any one of claims 4, wherein said filtration member has a multi-aperture structure with an aperture diameter ranged from 0.01 μm to 10 μm.

20. The body fluid sucking device, as recited in any one of claims 5, wherein said filtration member has a multi-aperture structure with an aperture diameter ranged from 0.01 μm to 10 μm.
